(19) United States
(12) Patent Application Publication
(10) Pub. No.: US 2023/0406818 A1
(43) Pub. Date: Dec. 21, 2023

(54) SYNTHESIS OF ALKYL BIS(FLUOROSULFONYL)IMIDE

(71) Applicant: UCHICAGO ARGONNE, LLC, Chicago, IL (US)

(72) Inventors: Trevor L. Dzwiniel, Carol Stream, IL (US); Krzysztof Z. Pupek, Plainfield, IL (US); Amber Tabaka, Downers Grove, IL (US)

(73) Assignee: UCHICAGO ARGONNE, LLC, Chicago, IL (US)

(21) Appl. No.: 18/205,395

(22) Filed: Jun. 2, 2023

Related U.S. Application Data
(60) Provisional application No. 63/354,171, filed on Jun. 21, 2022.

(51) Int. Cl. C07C 303/40

(52) U.S. Cl. CPC C07C 303/40

(57) ABSTRACT

Provided herein is a process comprising reacting a metal salt of bis(fluorosulfonyl)imide with an effective amount of di(C₁₋₃ alkyl) sulfate or di(C₂₋₃ alkenyl) sulfate in a solvent substantially free of dioxane to provide an N—(C₁₋₃ alkyl) or an N—(C₂₋₃ alkenyl) bis(fluorosulfonyl)imide, wherein the metal salt is an alkali or alkaline earth metal salt, the effective amount ranges from a molar excess to 10 molar equivalents of di(C₁₋₃ alkyl) or di(C₂₋₃ alkenyl) sulfate, and the solvent is selected from acyclic C₄₋₁₂ ethers and/or C₄₋₁₂ esters.

### CROSS-REFERENCE TO RELATED APPLICATIONS

This application claims priority to and the benefit of U.S. Provisional Patent Application No. 63/354,171, filed Jun. 21, 2022, the contents of which are incorporated by reference in their entirety.

### STATEMENT OF GOVERNMENT INTEREST

This invention was made with government support under Contract No. DE-AC02-06CH11357 awarded by the United States Department of Energy to UChicago Argonne, LLC, operator of Argonne National Laboratory. The government has certain rights in the invention.

### TECHNICAL FIELD

The present technology relates generally to synthesizing a solvent for use in lithium ion batteries, more particularly to synthesizing a non-flammable, non-carbonate solvent in a scalable manner without using highly toxic dioxane or an extreme excess of dimethyl sulfate.

### BACKGROUND

Electric batteries consist of one or more electrochemical cells that generate electrical energy using chemical reactions. Historically, many batteries utilized liquid electrolytes. Lithium-ion batteries (“LIB”) were developed as an alternative to these traditional batteries. LIBs have the advantage of being rechargeable and having higher energy densities relative to traditional electric batteries, low self-discharge, and minimal (if any) memory effect.

In LIB operation, lithium ions migrate through an electrolyte from the anode to the cathode during discharge. When the LIB recharges, the lithium ions migrate back through the electrolyte to the anode. One drawback to LIBs, though, is that they often use carbonate-based electrolyte solvents. These flammable solvents present safety hazards, especially if the battery is damaged.

LIBs utilizing non-flammable solvents are being explored. One such non-flammable solvent is N-methyl bis(fluorosulfonyl)imide (“Me-FSI”), which has shown promise for use in LIBs. Not only is this solvent safer than flammable solvents, but also has the potential to work at higher voltages relative to traditional solvents. However, traditional methods of preparing ME-FSI use dioxane and large quantities (e.g., 15-20 molar equivalents) of dimethyl sulfate as an alkylating agent and a solvent, both of which are toxic. Additionally, dioxane is difficult to remove from the Me-FSI product. Finally, the traditional methods of preparing Me-FSI utilize a highly exothermic quench of the dimethyl sulfate that pose significant safety hazards, especially as production is scaled to larger quantities. Moreover, the conventional methods suffer from modest Me-FSI yield and produce large quantities of impurities.

### SUMMARY OF THE TECHNOLOGY

The present technology provides improved processes of making N-alkyl or N-alkenyl bis(fluorosulfonyl)imide without the need for including toxic dioxane solvent or using, e.g., a large excess of toxic dialkyl sulfate to force the reaction to completion. The processes includes reacting a metal salt of bis(fluorosulfonyl)imide with an effective amount of di(C₁₋₃ alkyl) sulfate or di(C₂₋₃ alkenyl) sulfate in a solvent substantially free of dioxane to provide a C₁₋₃ alkyl or a C₂₋₃ alkenyl) bis(fluorosulfonyl)imide, wherein the metal salt is an alkali or alkaline earth metal salt, the effective amount ranges from a molar excess to 10 molar equivalents of di(C₁₋₃ alkyl) or di(C₂₋₃ alkenyl) sulfate, and the solvent is selected from acyclic C₄₋₁₂ ethers and/or C₄₋₁₂ esters.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values.

The phrase “and/or” as used in the present disclosure will be understood to mean any one of the recited members individually or a combination of any two or more thereof—for example, “A, B, and/or C” would mean “A or B or C; A and B; A and C; B and C; or the combination of A, B, and C.”

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “alkyl” groups include straight chain and branched alkyl groups having from 1 to 12 carbon atoms, e.g., 1 to 8 carbons, 1 to 6 carbons, 1 to 4 or, in some embodiments, from 1, 2 or 3 carbon atoms. Alkyl groups may be substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein the term haloalkyl is an alkyl group having one or more halo groups. In some embodiments, haloalkyl refers to a per-haloalkyl group. In any embodiments, the alkyl groups are unsubstituted alkyl groups.

Alkenyl groups are straight chain, branched or cyclic alkyl groups having 2 to about 20 carbon atoms, and further including at least one double bond. In some embodiments alkenyl groups have from 1 to 12 carbons, or, typically, from 1 to 8 carbon atoms. Alkenyl groups may be substituted or unsubstituted. Alkenyl groups include, for instance, vinyl, propenyl, 2-butenyl, 3 butenyl, isobutenyl, cyclohexenyl, cyclopentenyl, cyclohexadienyl, butadienyl, pentadienyl, and hexadienyl groups among others. Alkenyl groups may be substituted similarly to alkyl groups. In any embodiments, the alkenyl groups are unsubstituted alkenyl groups.

As used herein, the term “acyclic C₄₋₁₂ ethers includes ethers (a single ether oxygen) or di-ethers (two ether oxygens) or polyethers of straight or branched alkyl groups having 4 to 12 total carbon atoms, e.g., dibutyl ether, 1,2-dimethoxyethane, diethylene glycol dibutyl ether, or diethylene glycol butyl methyl ether.

As used herein, the term “C₄₋₁₂ esters” includes straight or branched alkyl esters of straight or branched alkanoic acids having 4 to 12 total carbon atoms, e.g., ethyl acetate, n-propyl acetate, isopropyl acetate, methyl propanoate and the like.

As used herein, the term “equivalents” means molar equivalents. That is, equivalents is the ratio of the mole amount of one compound in terms of a more limiting amount of another compound. For example, if K—FSI is the limiting reagent in a reaction, the amount of K—FSI would be considered 1 molar equivalent and any other reagents or reactants would be calculated as the molar ratio to the molar amount of K—FSI present. If, e.g., 0.5 moles of K—FSI is used and 2 moles of dimethyl sulfate are used in a reaction, one would have 4 equivalents of dimethyl sulfate per equivalent of K—FSI.

As used herein, the phrase “substantially free of dioxane” refers to a solvent or composition comprising less than 5 wt % dioxane. In some embodiments the solvent or compositions comprises less than 5%, less than 4%, less than 3%, less than 2%, less than 1%, less than 0.5 wt %, less than 0.2 wt %, less than 0.1 wt %, or less than 0.01 wt % dioxane. In any embodiments, the solvent is free of added dioxane.

The traditional method for synthesizing Me-FSI traditional suffers from a number of deficiencies, including low Me-FSI yield and high levels of impurities. The present technology addresses these deficiencies as well as provides additional advantages.

In one aspect, the present technology provides a process that includes reacting a metal salt of bis(fluorosulfonyl)imide with an effective amount of di(C₁₋₃ alkyl) sulfate or di(C₂₋₃ alkenyl) sulfate in a solvent substantially free of dioxane to provide an N—(C₁₋₃ alkyl) or an N—(C₂₋₃ alkenyl) bis(fluorosulfonyl)imide, wherein
the metal salt is an alkali or alkaline earth metal salt,
the effective amount ranges from a molar excess to 10 equivalents of di(C₁₋₃ alkyl) sulfate or di(C₂₋₃ alkenyl) sulfate, and
the solvent is selected from acyclic C₄₋₁₂ ethers and/or C₄₋₁₂ esters.

In the present methods, the reaction may be performed with an alkali metal or alkaline earth metal salt of bis(fluorosulfonyl)imide as a reactant. Thus, in any embodiments herein, the reactant may be lithium bis(fluorosulfonyl)imide, sodium bis(fluorosulfonyl)imide, potassium bis(fluorosulfonyl)imide, or any other alkali metal bis(fluorosulfonyl)imide. In certain embodiments, an alkaline earth metal salt of bis(fluorosulfonyl)imide may be used such as, but not limited to, calcium or magnesium bis(fluorosulfonyl)imide.

The present methods employ a di-(C₁₋₃ alkyl) sulfate to alkylate the alkali metal or alkaline earth metal salt of bis(fluorosulfonyl)imide and produce the C₁₋₃ alkyl FSI. In any embodiments of the present methods, e.g., any of dimethyl sulfate, diethyl sulfate or dipropyl sulfate (including, e.g., diisopropyl sulfate) may be used to produce the corresponding methyl-FSI, ethyl-FSI, or propyl-FSI. Typically, a molar excess of the di-(C₁₋₃ alkyl) sulfate is used to improve yields of the product, e.g., up to about 10 equivalents of the di-(C₁₋₃ alkyl) sulfate may be used. However, in some embodiments, less than 10 equivalents may be used such as about 2, about 3, about 4, about 5, about 6, about 7, about 8, or about 9 equivalents of the di-(C₁₋₃ alkyl) sulfate or a range between and including any two of the foregoing values. For example, about 2 to about 8 equivalents, about 3 to about 5 equivalents, or about 4 equivalents of di-(C₁₋₃ alkyl) sulfate may be used.

In any embodiments herein, the reaction may be performed in a solvent substantially free of dioxane. The solvent, upon heating dissolves the metal salt of bis(fluorosulfonyl)imide. The inventors have found very few solvents capable of simultaneously dissolving the metal salt, allowing use of a much smaller excess of di-(C₁₋₃ alkyl) sulfate without dioxane, and providing good yields of product. Suitable solvents for use in the present methods may be selected from the group consisting of acyclic C₂₋₆ ethers, C₂₋₆ esters, C₂₋₆ alkylene diols and any two or more thereof. Non-limiting examples of such solvents include 1,2-dimethoxyethane, ethyl acetate, isopropyl acetate, diethylene glycol dibutyl ether, diethylene glycol dimethyl ether, and combinations of any two or more thereof.

A range of concentrations of reactants may be used in the present methods. Typically, concentrations would be selected to achieve the highest yield at the highest conversion of the metal salt of bis(fluorosulfonyl)imide while limiting formation of side products and length of reaction. For example, the concentration of this imide may be about 0.1 molar (M) to about 10 M, and the concentration of di-(C₁₋₃ alkyl) sulfate or di-(C₂₋₃ alkenyl) sulfate similarly may be about from about 1 M to about 10 M. Those skilled in the art will understand that lower concentrations may be used but may lengthen the reaction time, and that higher concentrations may be used if the reactants are sufficiently soluble at the desired concentration. Thus, in any embodiments the concentration of the metal salt of bis(fluorosulfonyl)imide may be about 0.1, about 0.2, about 0.3, about 0.5 M, about 0.6 M, about 0.7 M, about 0.8 M, about 0.9 M, about 1 M, about 1.1 M, about 1.2 M, about 1.3 M, about 1.4 M, about 1.5 M, about 2 M, about 3 M, about 4 M, about 5 M, about 6 M, about 7 M, about 8 M, about 9 M, about 10 M, or in a range between and including any two of the foregoing values, e.g., 0.5 to 1.5 M. In any embodiments, the concentration of di-(C₁₋₃ alkyl) or di-(C₂₋₃ alkenyl) sulfate may be about 0.1 M, about 0.25 M, about 0.5 M, about 0.75 M, about 1 M, about 1.5 M, about 2 M, about 2.5 M, about 3 M, about 3.5 M, about 4 M, about 4.5 M, about 5 M, about 5.5 M, about 6 M, about 6.5 M, about 7 M, about 8 M, about 9 M, about 10 M, or in a range between and including any two of the forgoing values, e.g., 1.5 M to about 5 M.

In any embodiments of the methods herein, the reaction may be performed at a temperature from about 40° C. to about 100° C. Thus, in any embodiments herein, the reaction may be performed at a temperature of about 40° C., about 50° C., about 60° C., about 65° C., about 70° C., about 75° C., about 80° C., about 85° C., about 90° C., about 95° C., about 100° C., or a range between or including any two of the foregoing values. For example, the reaction may be performed at a temperature of about 70° C. to about 90° C. or from about 80° C. to about 85° C.

In any embodiments herein, a range of reaction times may be employed, depending on reaction scale and conditions. For example, the reaction time may range from about 1 hour to about twenty-24 or even 48 hours. Thus, in any embodiment herein, the reaction may be performed for about 1 hour, about 2 hours, about 3 hours, about 4 hours, about 5 hours, about 6 hours, about 8 hours, about ten hours, about 12 hours, about 14 hours, about 16 hours, about 18 hours, about 20 hours, about 22 hours, about 24 hours, about 30 hours, about 40 hours, about 48 hours, or any range between or including any two of the foregoing values.

In any embodiment herein, the reaction may be performed in a batch process, continuous process, or semi-batch process. In batch processes, materials are placed in reaction vessels at the start of the reaction and are only removed at the end. In continuous processes, materials flow into and out of the reaction vessel at a constant rate for the duration of the reaction time. Semi-batch processes are neither batch processes nor continuous processes. In semi-batch processes, reactants may be added and/or products may be removed periodically.

In any embodiments, the present methods may further include purifying the alkyl bis(fluorosulfonyl)imide from the reaction mixture. Generally, the products may be conveniently distilled to provide purified products. For example, Me-FSI may be distilled under reduced pressure or at atmospheric pressure. In any embodiments, two distillations may be used to purify product, e.g., one under reduced pressure and one at atmospheric pressure.

The present invention, thus generally described, will be understood more readily by reference to the following examples, which are provided by way of illustration and are not intended to be limiting of the present invention.

### EXAMPLES

### Reagents

1,4-Dioxane, anhydrous, 99.8%, Sigma Aldrich; Dimethyl Sulfoxide, 99.9%, VWR Chemicals; N,N-Dimethylformamide, ≥99.8%, VWR Chemicals; Butyl Acetate, ReagentPlus, 99.5%; Dimethyl sulfate, ≥99.5%, Sigma Aldrich; 1,2-Dimethoxyethane, ≥99.0%, TCI America; Lithium bis(fluorosulfonyl)imide, 100%, Arkema Innovative Chemistry, Propionitrile, purum, ≥99.0%; Lithium bis(fluorosulfonyl)imide (30%) in Ethyl Methyl Carbonate; Sulfuric Acid, ACS Reagent, 95-98%, Sigma Aldrich; Isopropyl Acetate, 99%, BeanTown Chemical; Potassium Bis(fluorosulfonyl)amide, 98%, Crysdot; Chloroform, ACS, >99.8%, VWR Chemicals; 18-crown-6 Ether, >98%, TCI; 1,2-Dichlorobenzene, HPLC grade, 99%, Sigma Aldrich; Ethyl Acetate, ≥99.9%, OmniSolv High Purity Solvent, Dimethyl Carbonate, ReagentPlus, 99%, Sigma Aldrich; Triethylene Glycol, 99%, Alfa Aesar; Diethylene glycol dibutyl ether, ≥99%, Sigma Aldrich; Diethylene glycol, 99%, Alfa Aesar; Toluene, Anhydrous, 99.8%, Sigma Aldrich; Acetonitrile, ≥99.5, VWR Chemicals; Diethylene glycol Butyl Methyl Ether, ≥99.0%, TCI.

### Example 1—Traditional Method for Synthesizing Me-FSI

General Procedure. The procedure of EP 2415758 Bi (to Honda et al.) was followed. In an inert atmosphere, 0.25 g (1.14 mmol) potassium bis(fluorosulfonyl)imide (K—FSI) and 0.098 mL (1 molar equivalent) anhydrous 1,4-dioxane were added to a 5 mL vial. Dimethyl sulfate (either 15 molar equivalents or 20 molar equivalents) was slowly added to the vial and the mixture was stirred at 100° C. for 3 h. The K—FSI completely dissolved when the solution temperature exceeded 40° C. At or above 100° C., precipitation began to occur. The reaction was monitored hourly using ¹⁹F-NMR, where DMSO and water were used to solubilize the sample prior to ¹⁹F-NMR analysis. Analytical data were consistent with methyl bis(fluorosulfonyl)imide as the product. See data in Example 5.

Results. Each method (using 15 molar equivalents and 20 molar equivalents of dimethyl sulfate) yielded significant amounts of product. In both methods, after 1 h about 80% of the starting material was converted to Me-FSI, with about 15% of the starting material being converted to a side product and the remaining starting material being unreacted. Me-FSI yield decreased to about 75% over the course of the next two hours while the amount of side-product increased, suggesting possible decomposition.

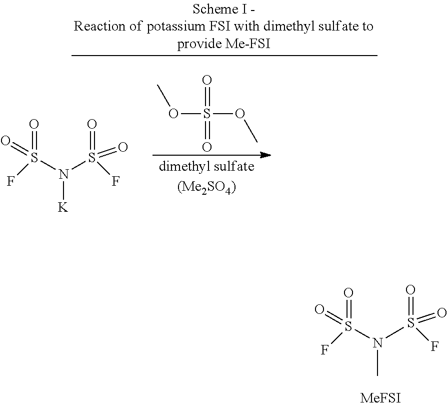

Purification. Following the reaction completion, the temperature was decreased to 70° C. Ice water was added slowly until the solution volume was doubled. The quenched reaction was stirred for 1 h and allowed to cool to room temperature. Me-FSI, which is denser than water separated from the mixture as the bottom layer. The aqueous layer was washed three times with chloroform to extract any Me-FSI that remained after the ice water wash. Another water wash was performed on the chloroform organic layer using 5 times the initial (reaction) volume of dioxane. Sodium sulfate (Na₂SO₄) was used to dehydrate the organic layers and then filtered. Chloroform was removed via rotary evaporation followed by distillation at 50-65° C. while under 50 torr for the complete removal of chloroform and residual dioxane. At the same pressure, the temperature is increased to 70-75° C. for the collection of Me-FSI. Final yield was approximately 30%.

Notably, this traditional Me-FSI synthesis procedure requires an excessive amount of dimethyl sulfate. Purification was inconsistent due to solubility issues.

### Example 2—Synthesis of Me-FSI without Dioxane or Other Solvents

Using Dimethyl Sulfate as Reaction Solvent. K—FSI (0.1 g) was dissolved in dimethyl sulfate. The amount of dimethyl sulfate was varied from 15 to 5 molar equivalents. In theory, only one molar equivalent should be necessary for full yield of product. These trials suffered from solubility issues, where the 10 molar equivalents trial had to be manually mixed very frequently for the first 2 hours. The 5 molar equivalents trial completely solidified within an hour and was unusable. Lowering the amount of dimethyl sulfate and eliminating the use of dioxane was not possible without incorporating a solvent.

### Example 3—Synthesis of Me-FSI Using K—FSI and Other Solvents

Screening Other Reaction Solvents. The general procedure of Example 1 was altered to add K—FSI to the selected solvent being screened for use. Where noted, 1,4-dioxane was added to the solvent solution and lastly, the dimethyl sulfate (5 molar equivalents) was slowly added. The solution was heated to an appropriate temperature (at or near the boiling point of solvent) and mixed for a number of hours (until the reaction reached completion based on ¹⁹F-NMR monitoring). Due to the solubility issues, the gelatinous mixtures from Example 2 were analyzed with different solvents to determine whether the salt (potassium sulfate and potassium methyl sulfate) could be dissolved. The only solvents that dissolved the salt were dimethylformamide (DMF), dimethyl sulfoxide (DMSO), and triethylene glycol dimethyl ether (TEG). Because DMF and DMSO reacted with dimethyl sulfate to form an undesired product, they were unsuitable solvents for the reaction.

Aprotic Solvents. Aprotic solvents having higher boiling points were screened. Using TEG resulted in side product formation but no Me-FSI. Diethylene glycol dibutyl ether (DEG-DBE), diethylene glycol butyl methyl ether (DEG-BME), and propionitrile (PROP) were analyzed as possible solvents. Reactions in these solvents were allowed to run until ¹⁹F-NMR showed signs of Me-FSI decomposition or an excessive side product formation. Unlike the original procedure, these reactions needed more than one hour to complete.

Ether and Ester Solvents. Ethyl acetate (EtOAc) and 1,2-dimethoxyethane (DME) were tested as solvents. Each of these solvents resulted in higher yields of Me-FSI than other solvents tested in Table 1. DME and EtOAc have lower boiling points than previously tested solvents but distillation could still be used for purification. Notably, dioxane was not necessary for the reaction to proceed when using EtOAc and DME as solvents.

TABLE 1
Summary of Solvent Screening Results using K-FSI.

| Reaction Conditions* | | | | | Product | | |
|---|---|---|---|---|---|---|---|
| | | Temper- | | Dioxane | | Side | |
| | Time | ature | Solvent | molar | K-FSI | Product | Me-FSI |
| Solvent | (h) | (° C.) | (mL/g) | equivalents | % | % | % |
| DEG-BME | 5 | 80 | 7 | 1 | 41 | 11.3 | 47.7 |
| DEG-DBE | 5 | 80 | 10 | 1 | 6.7 | 34.6 | 58.7 |
| PROP | 5 | 80 | 7 | 1 | 65.8 | 10.7 | 17 |
| DMSO | 6 | 80 | 10 | 1 | 100 | 0 | 0 |
| DMF | 6 | 80 | 10 | 1 | 100 | 0 | 0 |
| EtOAc | 24 | 75 | 7 | 0 | 18.34 | 10.06 | 71.59 |
| DME | 5 | 85 | 5 | 0 | 14.78 | 5.75 | 79.46 |

*Reactions run with 1 molar equivalent K-FSI and 5 molar equivalents dimethyl sulfate.

Side Product Removal. The side product is water soluble and can be removed by washing. A trial was performed with only filtering and distilling in place of the water and chloroform washes, and the side product distills out after the product along with K—FSI and dimethyl sulfate.

Screening Reaction Conditions with EtOAc Solvent. The reaction proceeds more slowly when using EtOAc as a solvent, with some reactions taking as long as 24 hours. The amount of solvent was applied to see if it altered the time it took for the greatest yield along with if the thickness of the solution would have any significant change. Lower solvent amounts resulted in a faster reaction. The reaction proceeded slightly slower in the absence of dioxane, which resulted in a slight increase of side product formation. This is still preferable due to dioxane being very toxic and hard to extract from the solution.

Screening Reaction Conditions with DME Solvent. DME resulted in the greatest yield of Me-FSI and the side product was approximately halved in comparison to EtOAc. These trials did seem to decompose after a certain yield was reached, but this occurred with any solvent using K—FSI. After approximately 80% yield, the reaction slowed greatly and decomposition seemed to occur. The conditions outlined in Trial 2 (see Table 2-85° C., 5 molar equivalents dimethyl sulfate, 5 hours) resulted in a high Me-FSI yield (79.46%) with a relatively short reaction time.

TABLE 2
Reaction conditions and products using DME solvent.

| | Reaction Conditions | | | | Product | | |
|---|---|---|---|---|---|---|---|
| | | Dimethyl | | | K-FSI | | |
| | Temper- | sulfate | Solvent | | starting | Side | Me-FSI |
| | ature | molar | molar | Time | material | Product | Product |
| Trial | (° C.) | equivalents | equiv. | (h) | % | % | % |
| 1 | 85 | 4 | 5 | 5 | 17.36 | 7.16 | 75.48 |
| 2 | | 5 | | 5 | 14.78 | 5.76 | 79.46 |
| 3 | | 4 | 7 | 22 | 18.76 | 7.47 | 73.77 |
| 4 | | 5 | | 28 | 10.99 | 5.50 | 83.52 |

### Example 4—Synthesis of Me-FSI Using Li-FSI and Other Solvents

Investigation of Different Starting Material. Li-FSI was used in place of K—FSI to investigate whether there is any significant difference in the starting material salt. The better solvents seen with K—FSI (i.e., EtOAc and DME) were used along with acetonitrile (MeCN), PROP and Isopropyl Acetate (IPAC). The initial results are shown in Table 3 below.

TABLE 3
Reaction conditions and products for using Li-FSI starting material.

| Reaction Conditions | | | | | Product | | |
|---|---|---|---|---|---|---|---|
| | Dimethyl | | | | Li-FSI | | |
| | sulfate | | Solvent | | starting | Side | Me-FSI |
| | molar | Temp. | molar | Time | material | Product | Product |
| Solvent | equivalents | (° C.) | equivalents | (h) | % | % | % |
| EtOAc | 3 | 75 | 5 | 24 | 25.06 | 9.95 | 64.99 |
| PROP | 5 | 90 | 7 | 5 | 56.21 | 25.79 | 18. |
| MeCN | 5 | 80 | 7 | 5 | 36.29 | 53.20 | 10.51 |
| IPAC | 5 | 85 | 7 | 24 | 6.00 | 10.96 | 83.03 |
| DME | 3 | 85 | 7 | 28 | 12.96 | 5.87 | 81.17 |

When the reaction was carried out in DME, the reaction time, side product yield, and Me-FSI yield were similar when using K—FSI and Li-FSI. As previously discussed, EtOAc could be used as a solvent with K—FSI starting material but the reaction proceeded more slowly than when DME was used as a solvent. Using Li-FSI as a starting material, with EtOAc as a solvent resulted in a lower yield relative to using DME as a solvent. Using propionitrile and acetonitrile as solvents resulted in a high formation of side product. Isopropyl acetate (IPAC) performed comparably to DME with Li-FSI— both solvents provided Me-FSI in approximately 80% yield, with around 10% of the starting salt remaining and formation of a modest amount of side product. Dimethyl sulfate variation trials were conducted with both DME and EtOAc to see if the starting salt could be used up completely. Increasing the amount of dimethyl sulfate up to 7 molar equivalents still provided approximately the same results with remaining salt in solution. Even when the maximum yield was reached and an additional 2 molar equivalents dimethyl sulfate was added, there was no significant change to the solution.

Varying Temperature and Dimethyl Sulfate Equivalents. DME was analyzed with increased temperature and dimethyl sulfate. Even with the increase in temperature, the results still remained the same.

Summary of Results. With both Li-FSI and K—FSI starting materials, DME was the favorable solvent due to shorter reaction times and lower amounts of dimethyl sulfate relative to other solvents. K—FSI needed slightly more dimethyl sulfate for similar yields to Li-FSI.

Investigation of Alternative Reaction Catalysts. Dioxane has traditionally been used as a catalyst for the production of Me-FSI. However, dioxane is highly toxic; moreover, dioxane's boiling point (101° C.) is close to that of Me-FSI (70-75° C. at 50 torr) and is therefore difficult to remove from the final product by distillation. An alternative catalyst, 18-crown-6 ether, was investigated but yielded slow reactions and only about a 20% Me-FSI yield.

Investigation of Alternative Methylating Agents. Methyl Triflate (MeOTf) was used in place of dimethyl sulfate. After heating the solution for 15 seconds, the solution turned a very dark color. ¹⁹F-NMR revealed a new undesired product being formed immediately with any heat. None of the desired Me-FSI product was observed.

### Example 5—Synthesis of Me-FSI Production Scale Up

Scaled to 5 g Reaction. Reactions using Li-FSI and K—FSI in DME were scaled to 5 g. For a slightly better yield, more dimethyl sulfate is needed when using K—FSI as a starting material. The solution was thick for the first 3 hours of stirring and had to be manually stirred occasionally. The 5 gram Li-FSI scale up synthesis had a slightly higher yield than K—FSI but also a higher amount of side product. Comparing these two syntheses, Li-FSI seems to be more stable if left alone longer than needed. A few trials indicated that with K—FSI, some decomposition may occur. The previous procedure required the solution be mixing at 70° C. when the water wash was being performed. This was to wash out the side product, dimethyl sulfate, as well as the solvent. Due to dimethyl sulfate having an exothermic reaction with water, the temperature had to be monitored to not increase a significant amount. With these washes, ice water was to be added but there was no increase of temperature due to the significantly lower amount of dimethyl sulfate used. Chloroform washes of 20 mL were performed and the solutions were dried with Na₂SO₄ and other solvents removed with rotary evaporation to extract the product (44° C. at 84 mbar). The organic layers were then distilled at 42 torr and 60° C. Fractions were collected up to full vacuum and 65° C. The rest of the solution consisted mostly of dimethyl sulfate, side product, and salt. Final distillations were performed to get the final yield at approximately 40-45% (1.5-2 g) for both syntheses.

120 g K—FSI Scale-Up Synthesis (chloroform wash). In an inert atmosphere, 120 g K—FSI was added to 692 mL (5 molar equivalents) DME in a 2 L 3-necked round bottom flask. Lastly, a slow addition of 207.65 mL of dimethyl sulfate (4 molar equivalents) was added via addition funnel. The solution was then stirred and heated at 80° C. for hours while monitoring the reaction rate periodically. After approximately six hours the yield was around 70%. Due to decomposition occurring after a certain time, the temperature was lowered to 70° C. to slow the reaction rate. By the next day, the yield was approximately 81% and almost complete. The temperature was then increased back to 80° C. until the final yield of product was 83.5% with approximately 8.5% salt left unused. The reaction temperature was then decreased to 70° C. and 750 mL of water was gradually added to the solution. Ice water was used if there was a significant increase in temperature. For this specific solution, there was no increase in temperature. The solution was stirred at 70° C. after 500 mL of water was added. The solution was then cooled to room temperature and the bottom organic layer (Me-FSI) was extracted. ¹⁹F-NMR revealed that a significant amount of K—FSI was still present in the organic layer, so another water wash was performed. With the aqueous layer, 3×300 mL chloroform washes were performed to extract any remaining Me-FSI product. The chloroform solution collected was dried with Na₂SO₄ and the chloroform was removed with rotary evaporation (44° C. at 84 mbar). ¹⁹F-NMR revealed that Me-FSI was still present to an extent in the chloroform solution removed by rotary evaporation. Another distillation was performed in order to extract as much product as possible. An additional 8-10 grams of product was extracted from the chloroform. The organic layers were then distilled at 42 torr and 60° C. Fractions were collected up to full vacuum and 65° C. The rest of the solution consisted mostly of dimethyl sulfate, side product, and salt. Final distillations were performed to get a final yield of 42 grams.

Analytical Data for Me-FSI. NMR spectra were run on a Magritek SpinSolve 80 Carbon benchtop spectrometer. ¹⁹F-NMR (neat, 75 MHz) 54.92 ppm (q, J=1.6 Hz). ¹H-NMR (neat, 80 MHz) 3.4 ppm (t, J=1.5 Hz). ¹³C-NMR (neat, 20 MHz) 38.99 ppm (s). GCMS (EI) using Agilent DB 5 MS column (30° C. for 5 minutes, ramp at 35° C./min to 250° C., hold at 250° C. for 1 minute) Rt 5.67 minutes (>99% purity); m/z=194.0 [M−H]⁺.

150 g K—FSI Scale-Up Synthesis (chloroform wash). The same synthesis as the 120 g scale up was performed on a 150 scale. K—FSI (150 g) was added to 865 mL DME (5 equiv) in a 2 L 3-necked round bottom flask. A volume of 259 mL dimethyl sulfate was slowly added via addition funnel and the same procedure was performed as in the 120 scale up. This yielded 48 g Me-FSI.

120 g K—FSI Scale-Up Synthesis (filter and distillation). The same procedure for 120 g K—FSI synthesis was repeated. However, instead of the water and chloroform washes, after the highest yield was reached, the reaction was cooled down to room temperature. The solution was then filtered and put into a 2 L 3-necked round bottom flask and distillation was performed. A majority of the DME was removed at 70 torr and 55° C. Different fractions were then obtained until the solution was at full vacuum (about 1 torr) and 65° C. After this, the remaining solution was mostly dimethyl sulfate, side product, and K—FSI. There is some product in this solution but the remaining product was difficult to extract. After a few more distillations, the final yield was approximately 48 g Me-FSI (49.3% yield).

Fractions were combined for a final distillation, which resulted in ≥99.5% purity. The excess DME was removed by distillation at 19.5 torr and 54° C. The product was removed via distillation at 17.9 torr and 54° C. Vacuum distillation was used due to decomposition of K—FSI in solution at higher temperature. The final fractions of pure product was ≥99.982% by GC/MS analysis. In order to obtain this final purity, an Oldershaw column was used. The final amount of pure Me-FSI was 104.56 g.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified. Finally, it will be understood that disclosure of one of the foregoing terms also discloses embodiments using any of the other two terms or their equivalents.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A process comprising reacting a metal salt of bis(fluorosulfonyl)imide with an effective amount of di(C₁₋₃ alkyl) sulfate or di(C₂₋₃ alkenyl) sulfate in a solvent substantially free of dioxane to provide an N—(C₁₋₃ alkyl) or an N—(C₂₋₃ alkenyl) bis(fluorosulfonyl)imide, wherein
the metal salt is an alkali or alkaline earth metal salt,
the effective amount ranges from a molar excess to 10 equivalents of di(C₁₋₃ alkyl) sulfate or di(C₂₋₃ alkenyl) sulfate, and
the solvent is selected from acyclic C₄₋₁₂ ethers and/or C₄₋₁₂ esters.

2. The process of claim 1, wherein the metal salt is potassium bis(fluorosulfonyl)imide or lithium bis(fluorosulfonyl)imide.

3. The process of claim 1, wherein the solvent is selected from the group consisting of ethyl acetate, isopropyl acetate, diethylene glycol dibutyl ether, diethylene glycol butyl methyl ether, 1,2-dimethoxyethane and combinations of two or more thereof.

4. The process of claim 1, wherein the solvent comprises 1,2-dimethoxyethane.

5. The process of claim 1, wherein the solvent comprises ethyl acetate.

6. The process of claim 1, wherein the reaction is performed at a temperature from about 40° C. to about 100° C.

7. The process of claim 1, wherein the reaction is performed at a temperature from about 75° C. to about 90° C.

8. The process of claim 1, wherein the reaction is performed at a temperature from about 80° C. to about 85° C.

9. The process of claim 1, wherein the effective amount of di-(C₁₋₃ alkyl) sulfate is from about 2 to about 8 equivalents relative to the metal salt of bis(fluorosulfonyl)imide.

10. The process of claim 1, wherein the effective amount of di-(C₁₋₃ alkyl) sulfate is from about 3 to about 5 equivalents relative to the metal salt of bis(fluorosulfonyl)imide.

11. The process of claim 1, wherein the effective amount is about 4 equivalents of di-(C₁₋₃ alkyl) relative to the metal salt of bis(fluorosulfonyl)imide.

12. The process of claim 1, wherein the metal salt of bis(fluorosulfonyl)imide is present at a concentration of from about 0.5 molar to about 1.5 molar.

13. The process of claim 1, wherein the reacting takes place over about 1 hour to about 48 hours.

14. The process of claim 1, wherein the reacting takes place over about 5 hours to about 15 hours.

15. The process of claim 1, where in the reaction is performed for a time period between about 8 hours and about 12 hours.

16. The process of claim 1, wherein an alkyl bis(fluorosulfonyl)imide is produced in a continuous process.

17. The process of claim 1, wherein an alkyl bis(fluorosulfonyl)imide is produced in a batch or semi-batch process.

18. The process of claim 1, further comprising purifying the C₁₋₃ alkyl bis(fluorosulfonyl)imide.

19. The process of claim 18, wherein the purifying comprised distilling the C₁₋₃ alkyl bis(fluorosulfonyl)imide.

20. The process of claim 2, wherein the wherein the effective amount of di-(C₁₋₃ alkyl) sulfate is from about 3 to about 5 equivalents dimethylsulfate relative to the metal salt of bis(fluorosulfonyl)imide, the solvent comprises 1,2-dimethoxyethane, and the reaction is performed at a temperature of about 75° C. to about 90° C.
